(19) United States
(12) Patent Application Publication
(10) Pub. No.: US 2023/0181023 A1
(43) Pub. Date: Jun. 15, 2023

(54) DEVICE FOR TREATMENT OF DYSPHAGIA

(71) Applicant: Phagenesis Limited, Manchester (GB)

(72) Inventor: Conor Mulrooney, Manchester (GB)

(21) Appl. No.: 17/817,536

(22) Filed: Aug. 4, 2022

Related U.S. Application Data
(63) Continuation of application No. 16/303,778, filed on Nov. 21, 2018, now abandoned.

(30) Foreign Application Priority Data
May 27, 2016 (GB) 1609425.2

(51) Int. Cl. A61B 1/267; A61B 1/00; A61N 1/36; A61N 1/05; A61B 1/005; A61B 1/05; A61B 1/273

(52) U.S. Cl. CPC A61B 1/267; A61B 1/00135; A61N 1/36042; A61N 1/0517; A61B 1/00089; A61N 1/0519; A61B 1/005; A61B 1/00087; A61B 1/05; A61B 1/273; A61N 1/05

(57) ABSTRACT

The present invention defines a device for the treatment of dysphagia comprising a flexible member mounting an imaging device for insertion into a patient and one or more electrodes proximate the imaging device for imparting electrical stimulation to the patient.

### CROSS-REFERENCE TO RELATED APPLICATIONS

This application is a continuation of U.S. patent application Ser. No. 16/303,778, filed Nov. 21, 2018, which is a national phase application filed under 35 U.S.C. § 371 of International Application No. PCT/GB 2017/051482, filed May 25, 2017, which claims priority of GB Patent Application 1609425.2 filed May 27, 2016. Each of these applications is incorporated by reference herein in its entirety.

### FIELD

The present invention relates to a device for treatment of dysphagia.

### BACKGROUND

The use of electrical pharyngeal stimulation to treat dysphagia is known. Various devices have been described in the literature to deliver such stimulation. One of the requirements for effective pharyngeal stimulation is that the electrodes should be placed in contact with patient tissue at a certain location within the pharyngeal region. Given that the internal anatomy of patients can vary this can present some technical challenges.

One of the possible approaches to reduce the challenge of correct positioning of stimulating electrodes is to have a means to visualise the stimulating catheter containing those electrodes during its placement and/or its use. This can be done using an endoscope such that the endoscope camera provides visual information to direct passage or final positioning of the treatment catheter. A problem with this approach however is that passage of two catheters simultaneously in a patient can be problematic. It is frequently the case that both nostrils cannot be used to pass catheters due to anatomical limitations, and oral passage of an endoscope or electrical stimulation catheter carries with it the risk of the patient biting and damaging the medical equipment or harming themselves. This presents an additional technical challenge.

Last but not least a limitation of the current state of the art is that the effect of stimulation on swallow function cannot be visualised in real time. This may mean that critical information relating to the responsiveness of the patient to the applied stimulation may be lost and opportunities to further optimise stimulation conditions may also be lost.

### SUMMARY

The present invention seeks to overcome these technical challenges or limitations by integrating the functionality of an endoscope with the functionality of an electrical stimulation catheter. This may take a number of different forms and the embodiments below are given by way of example only and should not be considered limiting.

A first embodiment of the invention provides a device for the treatment of dysphagia comprising a flexible member mounting an imaging device for insertion into a patient and one or more electrodes proximate the imaging device for imparting electrical stimulation to the patient.

Four particular advantages of this first embodiment are that; i) only a single device needs to be inserted into the patient to both image a target area of tissue and impart electrical stimulation to the target area of tissue, ii) that it allows the imaging device to direct the passage of the electrodes, iii) that final positioning of the electrodes can be judged relative to the patients' anatomy visible via the imaging device, and iv) that the flexible member may be used to actively bring the electrodes in contact with target tissues to ensure good electrical contact.

The one or more electrodes may be mounted on a sleeve, wherein the sleeve is positioned around the flexible member and movable relative thereto.

The provision of a sleeve located around the flexible member such that the sleeve is freely moveable along the length of the flexible member, and located on the sleeve one or more electrodes designed to deliver electrical stimulation. Within the body of the sleeve there are current carrying means to connect the electrodes to a source of electrical stimulation external to the patient.

An additional advantage of the sleeve is that the electrodes can be moved relative to the terminal end containing the camera and as such the two functions of visualisation and stimulation are capable of independent positioning optimisation.

The device may further comprise a dog leg structure extending from the flexible member beyond the imaging device.

Three additional advantages provided by the dog leg structure are; that the target tissues can be seen within the field of view, that the electrode position can be changed before or during treatment without movement of the imaging device or field of view, and that the effect of the stimulation can be assessed through visualisation of the specific area being stimulated and the behaviour of these tissues. These behaviours might include for example muscular contraction, protection of the airways or clearing of residues.

### FIGURES

FIG. 1 shows a first embodiment of a device for treating dysphagia;

FIG. 2 shows a second embodiment of a device for treating dysphagia; and

FIG. 3 shows a third embodiment of a device for treating dysphagia.

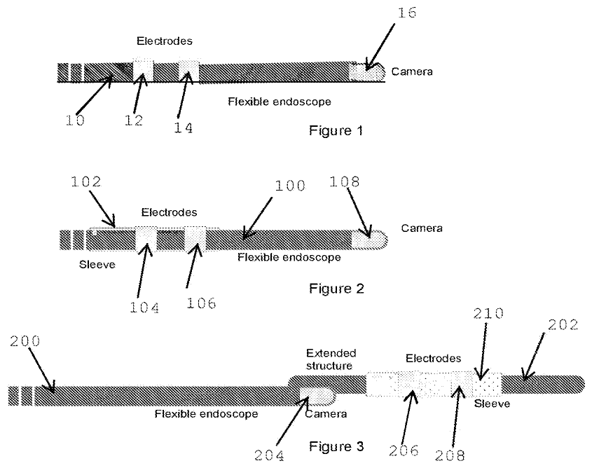

### DESCRIPTION

Embodiments of the invention will now be described by way of reference to the figures.

In a first embodiment of the invention there is a catheter (10), in the form of a flexible endoscope, with electrodes (12, 14) designed to deliver electrical stimulation to target tissues. The electrodes (12, 14) are integrated into the body of the endoscope (10) at a fixed position proximal an imaging device (16) part of the endoscope (10). The imaging device (16) part of the endoscope is positioned at an end of the endoscope (10). Within the body of the flexible endoscope (10) there are current carrying means to connect the electrodes (12, 14) to a source of electrical stimulation external to the patient.

In a second embodiment there is a catheter in the form of a flexible endoscope (100) having an imaging device (108) at one end thereof with a sleeve (102) located on it such that the sleeve (102) is freely moveable along the length of the endoscope (100), and located on the sleeve (102) are one or more electrodes (104, 106) designed to deliver electrical stimulation to target tissues. Within the body of the sleeve (102) there are current carrying means to connect the electrodes (104, 106) to a source of electrical stimulation external to the patient.

In a third embodiment there is a catheter in the form of a flexible endoscope (200) with an extended structure (202) distal to an imaging device (204) located at an end of the flexible endoscope (200), the extended structure (202) incorporating one or more electrodes (206, 208) designed to deliver electrical stimulation to target tissues. The electrodes (206, 208) in this extended structure (202) may be either fixed and integrated into said extended structure (202), or may be disposed on a sleeve (210—as shown in FIG. 3) that is freely moveable along the length of the extended structure (202), such movement being controllable externally. In the latter case the electrodes (206, 208) can then be moved relative to the position, and additionally within the field of view, of the imaging device (204). Within the body of the flexible endoscope (200) or the body of the sleeve (210) there are current carrying means to connect the electrodes to a source of electrical stimulation external to the patient.

1-11. (canceled)

12. A method of treating a patient suffering from dysphagia, the method comprising:
inserting a flexible member carrying an imaging device and an electrode into a pharynx of the patient such that the electrode is positioned at a treatment site within the pharynx;
visualizing the electrode at the treatment site with the imaging device; and
delivering electrical stimulation energy to the treatment site with the electrode.

13. The method of claim 12, wherein the electrode is movable relative to the imaging device along a length of the flexible member.

14. The method of claim 12, wherein the treatment site is a first candidate treatment site, the method further comprising moving the electrode from the first candidate treatment site to a second candidate treatment site spaced apart from the first candidate treatment site.

15. The method of claim 14, wherein moving the electrode from the first candidate treatment site to the second candidate treatment site occurs without movement of the imaging device.

16. The method of claim 14, wherein moving the electrode from the first candidate treatment site to the second candidate treatment site occurs after visualizing the electrode at the first candidate treatment site with the imaging device.

17. The method of claim 14, wherein moving the electrode from the first candidate treatment site to the second candidate treatment site occurs before delivering the electrical stimulation energy.

18. The method of claim 14, wherein moving the electrode from the first candidate treatment site to the second candidate treatment site occurs after visualizing the electrode at the first candidate treatment site and before delivering the electrical stimulation energy.

19. The method of claim 18, wherein the electrical stimulation energy is delivered to the second candidate treatment site.

20. The method of claim 14, wherein moving the electrode from the first candidate treatment site to the second candidate treatment site occurs while delivering the electrical stimulation energy.

21. The method of claim 14, wherein moving the electrode from the first candidate treatment site to the second candidate treatment site occurs after visualizing the electrode at the first candidate treatment site and while delivering the electrical stimulation energy.

22. The method of claim 12, further comprising moving the flexible member within the pharynx such that the electrode contacts tissues of the pharynx at the treatment site.

23. The method of claim 12, wherein the flexible member has a closed distal end.

24. The method of claim 12, wherein the imaging device is a camera.

25. The method of claim 12, further comprising visualizing a behavior of tissues of the pharynx at the treatment site in response to the electrical stimulation energy with the imaging device.

26. The method of claim 25, wherein the behavior comprises a muscular contraction.

27. The method of claim 25, wherein the behavior comprises protection of airways of the patient.

28. The method of claim 25, wherein the behavior comprises clearing of residues.

29. The method of claim 12, wherein the flexible member is inserted nasally into the pharynx.

30. The method of claim 12, wherein the flexible member is inserted via one nostril of the patient into the pharynx.
